(19) United States
(12) Patent Application Publication
(10) Pub. No.: US 2005/0152955 A1
(43) Pub. Date: Jul. 14, 2005

(54) Electrostatically self-assembled antimicrobial coating for medical applications

(76) Inventors: Jay R. Akhave, Claremont, CA (US); Jan E. Carr, Cleveland, OH (US); Jaime C. Grunlan, College Station, TX (US); Albert Lin, Cerritos, CA (US)

Correspondence Address: Heidi A. Boehlefeld;Renner, Otto, Boisselle & Sklar, LLP, Nineteenth Floor, 1621 Euclid Avenue, Cleveland, OH, 44115, US

(21) Appl. No.: 11/014,045

(22) Filed: Dec. 16, 2004

Related U.S. Application Data
(60) Provisional application No. 60/530,096, filed on Dec. 16, 2003.

(51) Int. Cl. A61L 15/00

(52) U.S. Cl. 424/445

(57) ABSTRACT

A electrostatically self-assembled coating having a biologically active agent incorporated therein is provided. More particularly, a wound dressing having an antimicrobial coating within the dressing construction wherein an antimicrobial agent is released from the dressing over a period of time is produced using a layer-by-layer deposition process.

This application claims the benefit of provisional application 60/530,096 filed on Dec. 16, 2003, which is hereby incorporated herein by reference in its entirety.

### BACKGROUND OF THE INVENTION

The present invention relates to an electrostatically self-assembled coating having a biologically active agent incorporated therein. More particularly, the invention resides in a wound dressing having an antimicrobial coating within the dressing construction wherein an antimicrobial agent is released from the dressing over a period of time.

### SUMMARY OF THE INVENTION

In accordance with the present invention, an electrostatically self-assembled coating having at least one antimicrobial agent incorporated therein is provided. In one embodiment, the invention is a multilayer antimicrobial coating on a flexible substrate comprising alternating layers of at least one cationic material and at least one anionic material, and having at least one biologically active agent complexed with the cationic or anionic material. The thickness of each of the cationic and anionic layers is less than about 200 nanometers. The cationic material may comprise a polycation and the anionic material may comprise a polyanion.

In one embodiment, a multilayer, biologically active coating on a thin film dressing is provided. Both the biologically active coating and the thin film dressing are substantially transparent.

The multilayer biologically active coating, in one embodiment, contains as the active agent silver ions. The silver ions can be used alone or in combination with a second active agent, such as cetrimide.

In one embodiment, the invention is directed to a wound dressing comprising a multilayer coating wherein the multilayer coating comprises alternating layers of at least one cationic material and at least one anionic material; and has at least one biologically active agent complexed with the cationic or anionic material; wherein the thickness of each of the cationic and anionic layers is less than about 200 nanometers.

### BRIEF DESCRIPTION OF THE DRAWINGS

FIG. 1 is a cross-sectional view of the substrate and alternating layers of a positively charged polymeric material including an antimicrobial agent and negatively charged polymeric material.

FIG. 2 is a cross-sectional view of the substrate and alternating blocks of biologically active bilayers and biologically inactive bilayers.

FIG. 3 is a cross-sectional view of an adhesive coated substrate onto which the biologically active film has been deposited.

FIG. 4 is a cross-sectional view of a hydrogel dressing containing a biologically active coating.

FIG. 5 is a cross-sectional view of a pattern-coated adhesive dressing containing a biologically active coating.

FIG. 6 is a cross-sectional view of a foam wound dressing.

FIG. 7 is a cross-sectional view of a multi-layer dressing of the present invention.

FIG. 8 is a cross-sectional view of a controlled release dressing.

FIG. 9 is a cross-sectional view of a multi-layer controlled release dressing.

FIG. 10 is a cross-sectional view of a multifunctional dressing.

FIG. 11 is a top view of a hydrogel dressing comprising an antimicrobial film according to the present invention.

FIG. 12 is a top view of a hydrocolloid dressing comprising an antimicrobial film according to the present invention.

### DETAILED DESCRIPTION OF THE INVENTION

Antimicrobial Coating:

The biologically active film of the present invention is prepared by the alternate adsorption film method, or layer-by-layer self-assembly method. With this method, alternating positively and negatively charged layers are deposited onto a base material or substrate by soaking or dipping the base material in a cationic solution and in an anionic solution until a multilayer film of the desired thickness is formed. Each individual layer has a thickness within the nanometer range. Specifically, the thickness of each deposited polymeric layer is generally less than about 200 nanometers. In one embodiment, the thickness is less than about 100 nanometers. In one embodiment, the thickness is of each layer is within the range of about 5 nanometers to about 60 nanometers. In another embodiment, the thickness of each layer is within the range of about 15 nanometers to about 50 nanometers.

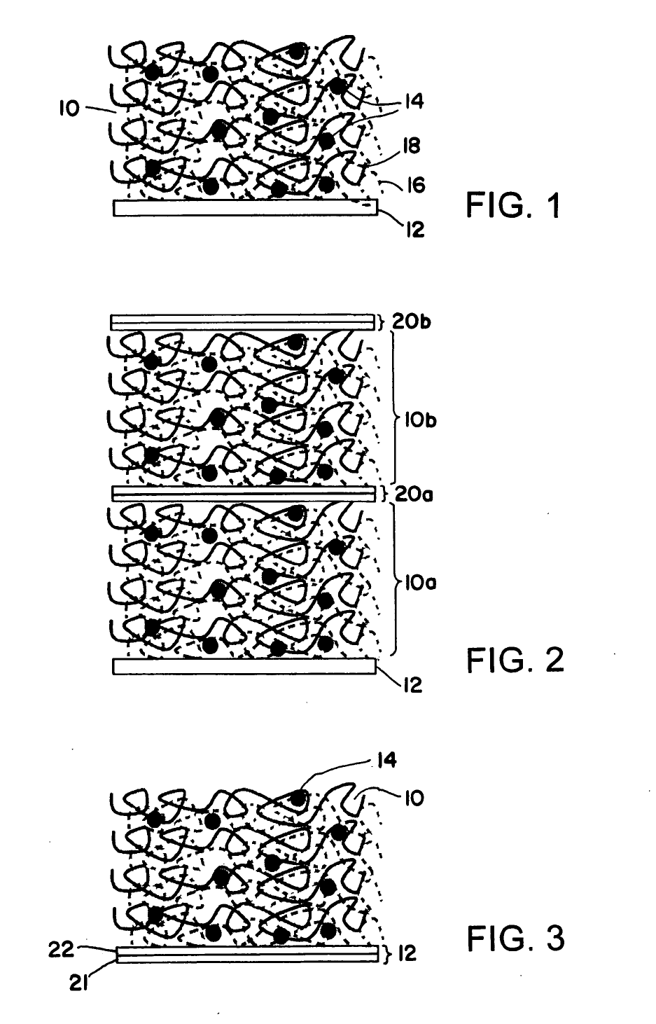

FIG. 1 (not to scale) illustrates the biologically active film of the present invention, in which biologically active coating 10 is deposited onto substrate 12. Biologically active coating 10 is made up of alternating layers of cationic polyelectrolyte 16 and anionic polyelectrolyte 18. In one embodiment, the coating comprises 2 to 100 bilayers of cationic and anionic polyelectrolytes. In another embodiment, the coating comprises 4 to 50 bilayers, and in yet another embodiment, 4 to 35 bilayers. Biologically active agent 14 is complexed with either the cationic or anionic layer, depending on the charge of the agent. As used herein, the term “complexed” means the biological agent is interconnected with, intermingled with, deposited with, dispersed within, and/or bonded to the polyelectrolyte. For example, if the biologically active agent were positively charged, such as silver ions, Ag⁺, the agent would be complexed with the cationic polyelectrolyte. The silver ions can be deposited simultaneously with the cationic polyelectrolyte.

The cationic and anionic layers are deposited onto the substrate from dilute solutions, typically aqueous, of polyelectrolytes. Polyelectrolytes, in general, are polymers with groups that are capable of ionic dissociation and may be a constituent or substituent of the polymer chain. The number of these groups capable of ionic dissociation in polyelectrolytes is normally so large that the polymers are water-soluble in the dissociated form (also called polyions). The term polyelectrolyte also means ionomers with which the concentrations of ionic groups are insufficient for water solubility, but which have significant charges to enter into self-assembly. In one embodiment, the concentration of the polyelectrolyte in solution is about 0.05% to about 1% by weight.

Depending on the nature of the groups capable of dissociation, polyelectrolytes are divided into polyacids and polybases. On dissociation of polyacids, there is formation of polyanions, with elimination of protons, that can be both inorganic and organic polymers. Polybases contain groups capable to take up protons, for example, by reaction with acids to form salts.

Useful polycations include polydiallyldimethyl ammonium chloride (PDDA), polyallylamine hydrochloride, and copolymers containing quaternary ammonium acrylic monomers. Examples of quaternary ammonium acrylic monomers include methacryloxyethyltrimethyl ammonium chloride, acryloxyethyl dimethylbenzyl ammonium chloride, methacryloxyethyl dimethylbenzyl ammonium chloride and acryloxyethyltrimethyl ammonium chloride. Polymers capable of hydrogen bonding, or hydrogen donors include polyethyleneimine (PEI), polyvinylimidazole, polylysine, poly-N-methyl-N-vinylacetamide, polyvinyl-pyrrolidone, polyvinyl alcohol, polyacrylamide and copolymers of aminoacrylates. The polymers can also become cationic at low pH due to protonation. Copolymers of acrylamide and acryloxytrimethylammonium chloride are particularly useful.

Substituted acrylamides and methacrylamides may be included into the copolymer in relatively small amounts. In large amounts, substituted acrylamides and methacrylamides adversely affect the solubility of the polycation.

In one embodiment, the cationic copolymer comprises a copolymer of acrylamide monomer and acryloxyethyltrimethyl ammonium chloride. In another embodiment, the cationic copolymer comprises a cationic acrylamide commercially available from Cytec under the trade name Superfloc C-491. In yet another embodiment, the cationic copolymer comprises a cation-modified polyvinyl alcohol commercially available from Kuraray under the designation CM-318.

The anionic layer is deposited onto the substrate from a dilute solution, typically aqueous, of polyanions. Polyanions are formed from the dissociation of polyacids. Examples of polyacids include polyphosphoric acid, polyvinylsulfuric acid, polyvinylsulfonic acid, polyvinylphosphonic acid, polyvinylphenylsulphuric acid, polyamino acid, polyglutamic acid, polymethacrylic acid, polyethylene sulphonic acid, poly(2-acrylamide-2-methyl-1-propanesulfonic acid) and poly(acrylic acid) (PAA). Examples of the corresponding salts include polyphosphate, polysulfate, polysulfonate, polyphosphonate, polyacrylate, polystyrene-sulfonic acid sodium salt, polyvinyl-sulfonic acid potassium salt, poly(sodium 4-styrenesulfonate) (PSS), and a polyamic acid salt (PAATEA).

Polyelectrolytes suitable for use in the present invention include biopolymers such as, for example, alginic acid, gum arabic, nucleic acids, pectins, proteins and others, and chemically modified biopolymers such as, for example, ionic or ionizable polysaccharides, for example carboxymethylcellulose, chitosan and chitosan sulfate, and ligninsulfonates.

It is possible to crosslink polyelectrolyte molecules within and/or between the individual layers, for example, by crosslinking amino groups with aldehydes. A further possibility is to use amphiphilic polyelectrolytes, for example amphiphilic block or random copolymers with partial polyelectrolyte characteristics. Such amphiphilic copolymers consist of units differing in functionality, for example acidic or basic units on the one hand, and hydrophobic units on the other hand, such as styrenes, dienes or siloxanes etc., which can be arranged as blocks or randomly distributed over the polymer. It is possible by using copolymers that change their structure as a function of the external conditions to control the permeability or other properties of the coating in a defined manner.

The release of the biologically active agent(s) can be controlled via the dissolution of the coating layers by using polyelectrolytes that are degradable under particular conditions, for example photo-, acid-, base- or salt-labile polyelectrolytes.

Biologically Active Agents:

The biologically active agent of the present invention may be an antibacterial agent, an antifungal agent, an analgesic agent, a tissue healant agent, a local anesthetic agent, an antibleeding agent, an enzyme or a vasoconstrictor, or any other biologically active agent. One or more biologically active agent may be combined in the coating of the present invention.

Where the biologically active agent is deposited onto the substrate in the negatively charged layer, the agent is an anionic agent. Examples of such anionic agents include those selected from antibacterials including fusidic acid, pseudomonic acid, Ceftriaxone (Rocephin); antifungals including nafcillin, Nystatin, and undecylenic acid; analgesics including salicylic acid, salicylsulfonic acid and nicotinic acid; and antibleeding agents including adenosine diphosphate. Such biologically active agents may be used in the form of their salts.

Further specific examples of anionic agents include the following:

(1) Fusidic Acid is also known as (Z)-16-(Acetyloxy)-3;α,11α-dihydroxy-29-nor-8α,9,13α, 14-dammara-17(20),24-dien-21-oic acid; 3α,11α,16γ-trihydroxy-29-nor-8α,9,13α,14-dammara-17(20),24-dien-21-oic acid 16-acetate; 3α,11α,16-trihydroxy-4α,8,14-trimethyl-18-nor-5α,8α,9,13α,14γ-cholesta-(20),24-dien-21-oic acid 16-acetate; 3α,11,16-trihydroxy-4,8,10,14-tetramethyl-17-(1′-carboxyisohept-4′-enylidene)cyclo-pentanoperhydrophenanthrene 16-acetate; and ramycin. Its sodium salt, sodium fusidate, is also known as ZN 6, Fucidine, Fucidina, Fucidine and Fucidin Intertulle.

(2) Pseudomonic Acids. A group of antibacterial antibiotics produced by Pseudomonas fluorescens NCIB 10586 that have unusual structural features. Four members of the group are known: pseudomonic acid A, the major component, pseudomonic acid B, the 3,4,5-trihydroxy analog of A (also referred to as pseudomonic acid 1), pseudomonic acid D, the 4-nonenoic acid analog of A; and pseudomonic acid C, in which the epoxide oxygen is replaced by a double bond.

Pseudomonic Acid A. Mupirocin. [2S-2α(E),3β,4β,5α[2R*,3R*-((1R*,-2R*)]]]-9-[[3-Methyl-1-oxo-4-[tetrahydro-3,4-dihydroxy-5-[[3-(2-hydroxy-1-methylpropyl)oxiranyl]methyl]-2H-pyran-2-yl]-2-butenyl]oxy]nonanoic acid; pseudomonic acid A; trans-pseudomonic acid; BRL-4910A; Bactoderm; Bactroban; Eismycin. C₂₆H₄₄O₉; mol wt 500.63. C 62.38%, H 8.86%, O 28.76%. Major component of the pseudomonic acids, q.v., an antibiotic complex produced by Pseudomonas fluorescens NCIB 10586.

Pseudomonic Acid C, C₂₆H₄₄O₈, [2S-[2α(E),3 β,4β,5α(2E,4S*,5R*)]]-9 [[3-methyl-1-oxo-4-tetrahydro-3,4-dihydroxy-5-(5-hydroxy-4-methyl-2-hexenyl)-2H-pyran-2-yl]-2-butenyloxy)nonanoic acid.

Pseudomonic acid D, C₂₆H₄₂O₉, [2S-[2α[E(E)],3β,4β,5α-[2R*,3R*(1 R*,2R*)]]-9-[[3-methyl-1-oxo-4-Tetrahydro-3,4-dihydroxy-5-[[3-(2-hydroxy-1-methylpropyl)oxiranyl]-methyl]-2H-pyran-2-yl)-2-butenyl]oxy)-4-nonenoic acid.

(3) Nafcillin is also known as 6-(2-Ethoxy-1-naphthamido)-3,3-dimethyl-7-oxo-4-thia-1-azabicyclo[3.2.0]he ptane-2-carboxylic acid; 6-(2-ethoxy-1-naphthamido)penicillanate; and 6-(2-ethoxy-1-naphthamido)penicillin. The sodium salt is also known as Naftopen and Unipen.

(4) Nystatin is also known as Fungicidin; Diastatin; CandioHermal; Mycostatin; Moronal; Nystan; Nystavescent; and O-V Statin.

(5) Undecylenic Acid, also known as 10-Undecenoic acid; 10-hendecenoic acid; 9-undecylenic acid; Declid; Renselin; and Sevinon.

(6) Salicylic Acid is also known as 2-Hydroxybenzoic acid.

(7) Salicylsulfuric Acid is also known as 2-(Sulfooxy)benzoic acid; salicylic acid, acid sulfate; and salicylic acid sulfuric acid ester.

(8) Nicotinic Acid is also known as 3-Pyridinecarboxylic acid; pyridine-γ-carboxylic acid; P.P. factor; pellagra preventive factor; antipellagra vitamin; niacin; Nicacid; Nicagin; Niconacid; Nicotinipca; Nicyl; Akotin; Daskil; Tinic; Nicolar; and Wampocap.

(9) Adenosine Diphosphate is also known as Adenosine 5′-(trihydrogen diphosphate); ADP; adenosine 5′-pyrophosphoric acid; 5′-adenylphosphoric acid; and adenosinediphosphoric acid.

Where the biologically active agent is deposited in the positively charged layer, the agent is a cationic agent. Examples of such cationic agents include those selected from anti-bacterials including chlorhexidine, Bacitracin, Chlortetracycline, Gentamycin, Kanamycin, Neomycin B, Polymyxin B, Streptomycin, and Tetracycline; antifungals including Amphotericin B, Clotrimazole, and Miconazole; tissue healants including cysteine, glycine and threonine; local anesthetics, e.g., Lidocaine; enzymes including trypsin, Streptokinase, plasmin (Fibrinolysin) and Streptodornase; deoxyribonuclease; and cationic vasoconstrictors including epinephrine and serotonin. Such biologically active agents may be used in the form of their salts.

Further specific examples of such cationic agents include the following:

(1) Chlorhexidine, also known as N,N″-Bis(4-chlorophenyl)-3,12-diimino-2,4,11,13-tetraazatetradecanediimidamide; 1,1′-hexamethylenlenebis[5-(p-chlorophenyl)biguanide]; 1,6-bis[N′-(p-chlorophenyl)-N⁵-biguanido]hexane; 1,6-bis(N⁵-p-chlorophenyl-N′-diguanido)hexane; 1,6-di(4′-chlorophenyldiguanido) hexane; 10,040; Hibitane; Nolvasan; Rotersept; and Sterilon. Its gluconate is known as Hibiscrob.

(2) Bacitracin, also known as Ayfivin; Penitracin; Zutracin; and Topitracin.

(3) Chlortetracycline, also known as 7-Chloro-4-dimethylamino-1,4,4a,5,5a,6,11,12a-octahydro-3,6,10,12,12a-pentahydroxy-6-methyl-1,11,-dioxo-2-naphthacene carboxamide; 7-chlorotetracycline; Acronize; Aureocina; Aureomycin; Biomitsin; Biomycin; and Chrysomykine.

(4) Gentamycin includes Gentamicin C₁₈, which is also known as 0-3-Deoxy-4-C-methyl-3-(methylamino)-γ-L-arabinopyranosyl(1→6)-0[2,6-dramino-2,3,4,6-tetradeoxy-α-D-erythro-hexo pyranosy 1-(1→4)]-2-deoxy-D-streptamine and as gentamicin D.

Gentamicin A is also known as 0-2-Amino-2-deoxy-α-D-glucopyranosyl-(1→4)-O-[3-deoxy-3-(methylamino)-α-D-xylopyranosyl(1→6)]-2-deoxy-D-streptamine.

The C complex sulfate is also known as Cidomycin, Garamycin, Garasol, Gentalyn, Genticin, Gentocin, Refobacin, and Sulmycin.

(5) Kanamycin includes: Kanamycin A sulfate, also known as Cantrex, Cristalomicina, Kamycin, Kamynex, Kanacedin, Kanamytrex, Kanasig, Kanicin, Kannasyn, Kantrex, Kantrox, Otokalixin, Resistomycin (Bayer), Opthalmokalixan, Kantrexil, Kano, Kanescin, and Kanaqua; Kanamycin B, is also known as NK 1006, bekanamycin, and aminodeoxykanamycin; and Kanamycin B sulfate, also known as Kanendomycin, and Kanamycin.

(6) Neomycin is also known as Mycifradin; Myacyne; Fradiomycin; Neomin; Neolate; Neomas; Nivemycin; and Vonamycin Powder V. It also includes Neamine, which includes: Neomycin A, and Neomycin B, which is also known as Framycetin, Enterfram, Framygen, soframycin, Actilin, and antibiotique E.F.185. Neomycin B sulfate is also known as Fraquinol, Myacine, Neosulf, Neomix, Neobrettin, and Tuttomycin.

(7) Polymyxin includes: Polymyxin B, which is a mixture of polymyxins B. and B₂; Polymyxin B sulfate, which is also known as Aerosporin; Polymyxin B₁; Polymyxin B₁ hydrochloride; Polymyxin B₂; Polymyxin D₁; Polymyxin D₂; and Polymyxin E, which is also known as Colistin; Colimycin; Coly-Mycin; Totazina; and Colisticina.

(8) Streptomycin is also known as 0-2-Deoxy-2-(methylamino)-α-L-glucopyranoxy]-(1→2)-O-5-deoxy-3-C-formyl-α-L-lyxofurano-syl(1→4)-N,N′-bis(aminoiminomethyl)-D-streptamine; and streptomycin A. Its sesquisulfate is also known as streptomycin sulfate, Agristrep, Streptobrettin, Streptorex, and Vetstrep. Streptomycin B is also known as Mannosidostreptomycin; and mannosylstreptomycin.

(9) Tetracycline is also known as 4-(Dimethylamino)-1,4-4a,5,5a,6,-11, 12a-octahydro-3,6,10,12,12a-pentahydroxy-6-methyl-1,-11-dioxo-2-naphthacene carboxamide; deschlorobiomycin; tsiklomitsin; Abricycline; Achromycin; Agromicina; Ambramicina; Ambramycin; Bio-Tetra; Bristaciclina; Cefracycline suspension; Criseo-ciclina; Cyclomycin; Democracin; Hostacyclin; Omegamycin; Panmycin; Polycycline; Purocyclina; Sanclomycine; Steclin; Tetrabon; Tetracyn; Tetradecin. Its hydrochloride is also known as Achro, Achromycin V, Ala Tet, Ambracyn, Artomycin, Cefracycline tablets, Cyclopar, Diacycline, Dumocyclin, Fermentmycin, Mephacyclin, Partrex, Quadracycline, Quatrex, Ricycline, Rocyc-line, Stilciclina, Subamycin, Sustamycin, Teline, Telotrex, Tetra-bid, Tetrachel, Tetracompren, Tetra-D, Tetrakap, Tetralution, Tetramavan, Tetramycin, Tetrosol, Totomycin, Triphacyclin, Unicin, and Unimycin. Its phosphate complex is also known as Panmycin Phosphate, Sumycin, Tetradecin Novum, Tetrex, and Upcyclin. Its lauryl sulfate is known as Lauracycline.

(10) Amphotericin B is also known as Fungizone; Fungilin; and Ampho-Moronal.

(11) Clotrimazole is also known as 1-(2-Chlorophenyl)diphenyl-methyl]-1H-imidazole; 1-(o-chloro-α,α-diphenylbenzyl)imidazole; 1-[α-(2-chlorophenyl)benzldryl)imidazole; 1-[(o-chlorophenyl(diphenylmethylimidazole; dipheny-(2-chlorophenyl)-1-imidazolylmethane; 1-(o-chlorotrityl)imidazole; FB 5097; BAY b 5097; and Canesten; Lotrimin; Mycosporin.

(12) Miconazole is also known as 1-[2-(2,4-Dichlorophenyl)-2-[(2,4-dichlorophenyl)methoxyethyl]-1H-imidazole; and 1-[2,4-dichloro-γ-[(2,4-dichlorobenzyl-oxy]phenethyl]imidazole. Its nitrate is also known as R-14889, Albistat, Brentan, Conofite, Daktarin, Dermonistat, Epi-Monistat, Gyno-Daktarin, Gyno-Monistat, Micatin, and Monistat.

(13) Cysteine, Cys (IUPAC abbrev.) is also known as OL-cysteine; γ-mercaptoalanine; 2-amino-3-mercaptopropanoic acid; 2-amino-3-mercaptopropionic acid; and α-amino- -thiolpropionic acid.

(14) Glycine, Gly (IUPAAC abbrev.), is also known as aminoacetic acid; aminoethanoic acid; glycocoll; and Glycosthene.

(15) Threonine, Thr (IUPAC abbrev.), is also known as 2-amino-3-hydroxybutyric acid; α-amino-γ-hydroxybutyric acid; and 2-amino-3-hydroxybutanoic acid.

(16) Lidocaine is also known as 2-(Diethylamino)-N-(2,6-dimethylphenyl)acetamide; 2-diethylamino-2′,6′-acetoxylidide; α-diethylamino-2,6-dimethylacetanilide; lignocaine; Xylocalne; Xylotox; Leostesin; Rucaina; Isicaine; Duncaine; Xylestesin; Anestacon; Gravocain; Lidothesin; and Xylocitin.

(17) Fibronolysin is also known as Plasmin; serum tryptase; Actase; and Thrombolysin.

(18) Epinephrine is also known as 4-[1-Hydroxy-2-(methylamino)-ethyl]-1,2-benzenediol; 3,4-dihydroxy-α-[(methylamino)methyl]-benzyl alcohol; 1-(3,4-dihydroxyphenyl)-2-(methylamino)ethanol; 3,4-dihydroxy-1-[1-hydroxy-2-(methylamino)-ethylbenzene; methyl-aminoethanolcatechol; and adrenalin.

(19) Serotonin is also known as 3-(2-aminoethyl)-1H-indol-5-ol; 5-hydroxytryptamine; 3-(γ-aminoethyl)-5-hydroxyindole; 5-hydroxy-3-(γ-aminoethyl)indole; enteramine; thrombocytin; thrombotonin; and 5-HT.

(20) Metal salts, or like compounds with antibacterial metal ions, e.g., copper, silver, gold, platinum, zinc, tin, antimony and bismuth, and optionally with nonmetallic ions of antibacterial properties.

(21) Quaternary ammonium compounds, e.g., cetrimide, domiphen bromide, and polymeric quaternaries.

A particularly useful antimicrobial agent is Ag⁺. The silver ion is derived from a suitable silver salt, including silver bromide, silver fluoride, silver chloride, silver nitrate, silver sulfate, silver alkylcarboxylate, silver sulphadiazine or silver arylsulfonate.

Other biologically active agents include those disclosed in “Biochemistry of Antimicrobial Action” by T. J. Franklin and G. A. Snow, 4^{th} Edition, Chapman and Hall, 1981, incorporated herein by reference.

The biologically active coating may contain two or more active agents. In one embodiment, for example, the coating contains silver ions and cetrimide. Both the silver ions and the cetrimide can be deposited simultaneously in the cationic layers. Alternatively, silver ions can be deposited in one or more of the cationic layers, and cetrimide can be separately deposited in one or more other cationic layers of the biologically active coating.

In one embodiment, the multilayer coating is substantially transparent. The multilayer coating can be deposited onto a substantially transparent substrate, for example, a thin film dressing. The underlying wound can then be monitored without removing the dressing.

Inactive Barrier Layers:

In one embodiment of the invention, the biologically active coating contains inactive barrier layers within the coating structure. For example, the coating can comprise blocks of biologically active bilayers and blocks of inactive bilayers. FIG. 2 illustrates a biologically active film on a substrate in which the film includes biologically active bilayer blocks 10a and 10b and inactive bilayer blocks 20a and 20b. The blocks of biologically active bilayers are made up of alternating positively charged and negatively charged layers having a biologically active agent or agents in at least one of the positively charged and negatively charged layers. The blocks of inactive bilayers are made up of alternating positively charged and negatively charged layers having no biologically active agents in either the positively charged or negatively charged layers. The inactive bilayers can facilitate sustained release of the biologically active agent(s) by impeding the rapid diffusion of the active agent through the coating.

The inactive bilayers may comprise the cationic polyelectrolytes and anionic polyelectrolytes described above. Alternatively, the positively charged layer may comprise cationic polyelectrolytes and the negatively charged layer may comprise an inorganic material. Examples of inorganic materials include negatively charged platelets having a thickness of less than about 10 nanometers. Useful inorganic material includes platelet clays that are easily exfoliated in aqueous or polar solvent environments. The clays may be naturally occurring or synthetic. Platelet clays are layered crystalline aluminosilicates. Each layer is approximately 1 nanometer thick and is made up of an octahedral sheet of alumina fused to 2 tetrahedral sheets of silica. These layers are essentially polygonal two-dimensional structures, having a thickness of 1 nanometer and an average diameter ranging from 30 to 2000 nanometers. Isomorphic substitutions in the sheets lead to a net negative charge, necessitating the presence of cationic counter ions (Na+, Li+, Ca++, Mg++, etc.) in the inter-sheet region. The sheets are stacked in a face-to-face configuration with inter-layer cations mediating the spacing. The high affinity for hydration of these ions allows for the solvation of the sheet in an aqueous environment. At sufficiently low concentrations of platelets, for example less than 1% by weight, the platelets are individually suspended or dispersed in solution. This is referred to as “exfoliation”.

Useful clays are those belonging to the smectite family of clay, including montmorillonite, saponite, beidellite, nontronite, hectorite, laponite fluorohectorite and mixtures of these. A particularly useful clay is montmorillonite. This clay is usually present in a sodium ion exchange form. Montmorillonite clay is commercially available from Southern Clay Products, Inc. under the trade name Cloisite. In one embodiment, the clay comprises sodium montmorillonite.

Other useful inorganic materials in platelet form include layered titanates, including those within the chemical formula Ti_{1-δ}O₂^{4δ-}; layered perovskites, including HCa₂Nb₃O₁₀, HSrNb₃O₁₀, HLaNb₂O₇ and HCaLaNb₂TiO₁₀; and mica.

Substrates:

The substrate onto which the antimicrobial coating is deposited may be any substrate that the cationic material can be adsorbed directly, or indirectly with the aid of an adhesion promoter or tie layer. The substrate may be a polymeric material, metal, glass, fabric, a ceramic material, a crystalline material, or a multilayer substrate of one or more of these materials. In one embodiment, the substrate is optically transparent. The substrate may be rigid, or may be flexible.

When the coating is to be used in a wound dressing, the substrate must be sufficiently conformable to conform to the contours of skin to which it will be applied. The film may be porous, non-porous, woven or nonwoven or a foam film. The substrate may be chosen from, for example, non-woven meshes; woven meshes of fiberglass or acetate; gauze; polyurethane foams; polymeric films including polyolefins (linear and branched), halogenated polyolefins, polyamides, polystyrenes, nylon, polyesters, polyester copolymers, polyurethanes polysulfones, styrene-maleic anhydride copolymers, styrene-acrylonitrile copolymers, ionomers based on sodium or zinc salts of ethylene methacrylic acid, polymethyl methacrylates, cellulosics, acrylic polymers and copolymers, polycarbonates, polyacrylonitriles, and ethylene-vinyl acetate copolymers; composite wound dressings, and adhesive-coated, thin-film dressings.

The substrate may be an untreated film that is amenable to adsorption. Alternatively, the film may be treated by first exposing the film to an electron discharge treatment at the surface, e.g., corona treatment. Other surface treatments to enhance the adsorption of the cationic organic material are well known. For example, the surface of the substrate may be plasma treated, chemically treated or solvent washed. Additionally, polymeric films that have been pretreated to promote adhesion are commercially available. Examples of such pretreated films include the PET films available from DuPont Teijin Films under the designation ST504 (one side treated) and ST505 (both sides treated).

In one embodiment, the surface of the substrate is roughened to improve adhesion and to increase the surface area of the substrate surface. With increased surface area, such as with roughened surfaces and foamed substrates, the activity of the antimicrobial agent is increased.

The substrate can be a single-layered film or it can be a multi-layered construction. The multi-layered construction can be, for example, coextruded films and laminated films. The multi-layered constructions have two or more layers, and in one embodiment, two to about seven layers, and in one embodiment, about three to about five layers. The layers of such multi-layered constructions and polymer films can have the same composition and/or size or they can be different. The substrate can have any thickness that is suitable for the intended use of the antimicrobial article. In one embodiment the thickness of the substrate may be in the range of about 0.3 to about 20 mils, and in another embodiment, about 0.3 to about 10 mils, and in yet another embodiment about 0.5 to about 7 mils, and in a further embodiment about 1 to about 5 mils. The substrate can also be a foam sheet having a thickness of up to 2 inches, or 1.5 inches, or 1.25 inches or 1 inch.

In one embodiment of the present invention, the substrate onto which biologically active coating is deposited is a thin film dressing. Examples of thin film dressings are those described in U.S. Pat. Nos. 6,346,653; 6,066,773; 6,043,406; 5,762,620; 5,520,629; 5,501,661; 5,489,262 and 5,437,622, all of which are incorporated by reference herein. Generally, thin film wound dressings comprise a multilayer configuration having an upper cover sheet, an adhesive layer and a bottom carrier or liner. The liner is removed for application of the dressing to the patient. The thin film dressing is flexible in order to conform to the contour of the patient. The thin film dressing may be transparent for improved monitoring of the wound site. An absorbent material may be positioned on the adhesive layer. The absorbent material can be an absorbent pad placed in the middle of the dressing so that the pad is surrounded by adhesive for sufficient adhesion to the patient. Alternatively, the absorbent material is a hydrogel that is positioned on the adhesive layer or positioned directly on the upper cover sheet. Absorbent hydrogels and hydrogel adhesives are known in the art.

The absorbent material itself may contain medication, for example, an antibiotic, a healing promoting agent, an anti-inflammatory agent, a transdermal diffusable pharmaceutical, a coagulant or an anti-coagulant.

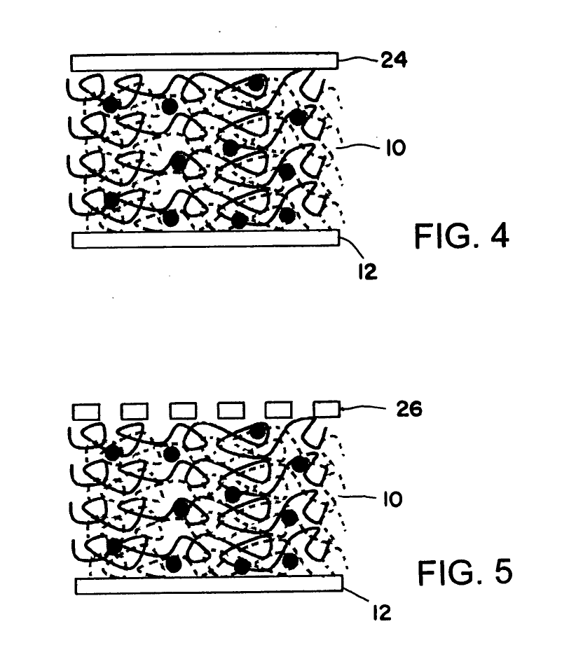

In one embodiment of the present invention, a hydrogel layer is applied to the biologically active film. This configuration is particularly useful as a wound dressing. FIG. 4 shows biologically active film 10 applied to substrate 12. Hydrogel layer 24 is applied over the biologically active film 10, so that the hydrogel is in direct contact with the patient's skin. Substrate 12 can be any flexible film. In one embodiment, substrate 12 is the cover sheet of the thin film dressing.

In another embodiment, illustrated in FIG. 5, an adhesive layer 26 is pattern-coated over the biologically active film 10 that is deposited onto substrate 12. Useful adhesives are any known medical grade adhesives. The medical adhesives include suitable acrylic based pressure sensitive adhesives (PSAs), suitable rubber based pressure sensitive adhesives and suitable silicone pressure sensitive adhesives.

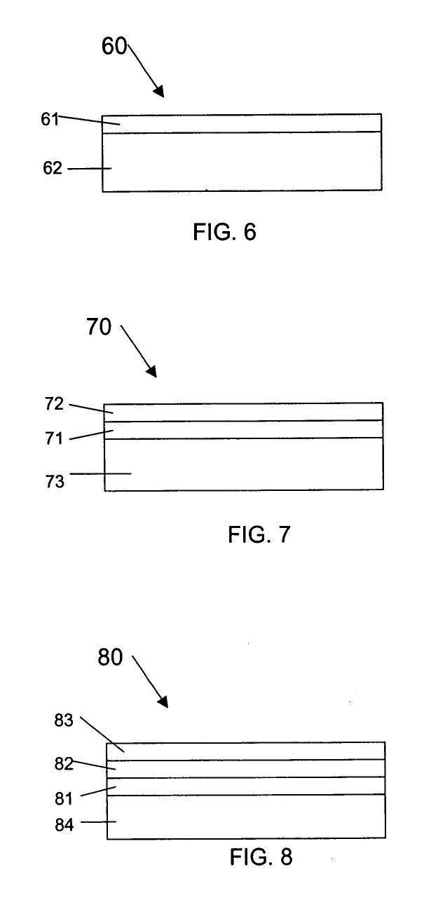

In one embodiment, illustrated in FIG. 6, a wound dressing 60 comprises an antimicrobial coating 61 on a polymeric foam substrate 62. The substrate 62 may comprise a polyurethane foam. The coating 61 comprises about 4 to about 32 bilayers of PEI with Ag+ layers alternating with PAA layers. The concentration of Ag+ in each cationic layer is about 1 mM to about 100 mM, or about 2 mM to about 20 mM. The overall thickness of the antimicrobial coating 61 is less than 1 micron. The Ag+ coating of the dressing is effective against the activity of S. aureus, E. coli, MRSA, VRE, P. aeruginosa, C. albicans, E. faecalis, S. pyogenes, C. perfringens, Klebsiella pneumoniae and E. faecium.

In one embodiment similar to that described with reference to FIG. 6, a wound dressing comprises a coating of alternating layers of PEI with cetrimide as the cationic layers and PAA as the anionic layers on a foam substrate. The number of bilayers is about 4 to about 32. The concentration of the cetrimide in the cationic layer is about 1 mM to about 100 mM, or about 2 mM to about 20 mM.

In one embodiment, the coating on the wound dressing comprises alternating bilayers of (a) PEI/Ag+ and PAA layers and (b) PEI/cetrimide and PAA layers. The total number of bilayers is about 4 to about 32.

In another embodiment, illustrated in FIG. 7, a wound dressing 70 comprises a first antimicrobial coating 71 on foam substrate 73 and a second antimicrobial coating 72 overlying the first coating 71. The first coating 71 comprises about 8 bilayers of PEI with Ag+ layers alternating with PAA layers. The concentration of Ag+ in the cationic layers is the same as that of the Ag+ layer of the coating 61 described above. The second coating 72 comprises about 8 bilayers of cetrimide layers alternating with PAA layers.

In one embodiment, illustrated in FIG. 8, a controlled release wound dressing 80 comprises a first antimicrobial coating 81 of alternating layers of PEI/Ag+ and PAA on substrate 84, an intermediate coating 82 of alternating layers of PEI and PAA overlying coating 81, and a second antimicrobial coating 83 of alternating layers of cetrimide and PAA overlying coating 82. The total number of bilayers is about 4 to about 35. In one embodiment, the number of bilayers in coating 81 is about 8. The number of bilayers in coating 82 is about 4 and the number of bilayers of coating 83 is about 4.

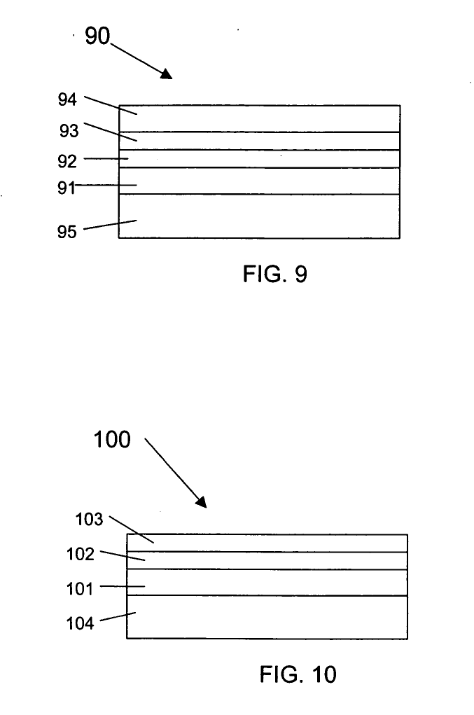

In one embodiment, illustrated in FIG. 9, a controlled release wound dressing 90 comprises a first antimicrobial coating 91 of alternating layers of PEI/cetrimide and PAA on substrate 95, an antibiotic layer 92 of alternating layers of PEI and PAA/antibiotic component overlying coating 91, an intermediate layer 93 of alternating layers of PEI and PAA overlying coating 92, and a second antimicrobial layer 94 of alternating layers of PEI/Ag+ and PAA overlying coating 93.

In another embodiment, a biocompatible coating is formed on a substrate. The biocompatible coating comprises alternating layers of chitosan and PSS on a substrate. The concentration of chitosan in the cationic layer and PSS in the anionic layer is about 0.1% to about 0.3% by weight. The number of bilayers is about 2 to about 20, or about 2 to about 8.

In one embodiment, illustrated in FIG. 10, a multifunctional, multi-layer dressing 100 comprises a biocompatible component and a controlled release antimicrobial component on a substrate 104. An antimicrobial coating 101 comprising alternating layers of PEI/Ag+ and PAA are formed on substrate 104. An intermediate coating 102 comprising alternating layers of PEI and PAA overlies coating 101. A biocompatibility layer 103 comprising alternating layers of chitosan and PSS overlies coating 102. The total number of bilayers is about 3 to about 35. In one embodiment, coating 101 comprises about 8 bilayers, coating 102 comprises about 4 bilayers and coating 103 comprises about 4 bilayers. Additional bilayers of PEI/Ag+ and PAA may be coated onto the biocompatibility layer 103.

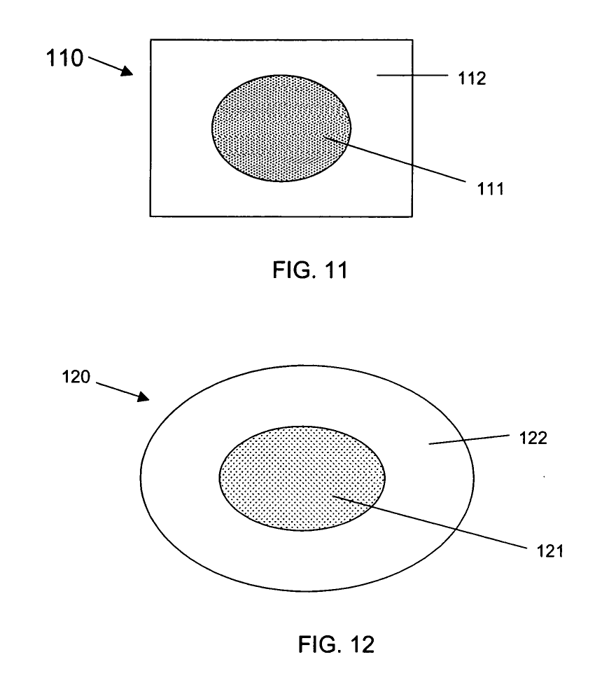

In one embodiment, illustrated in FIG. 11, a hydrogel dressing 110 comprises an antibacterial component 111 and a hydrogel contact component 112. The antibacterial component 111 comprises a substrate coated with alternating layers of PEI/Ag+ and PAA. The hydrogel contact component 112 comprises a hydrogel such as those known in the art as being particularly useful in wound dressings.

In another embodiment, illustrated in FIG. 12, a hydrocolloid dressing 120 comprises an antibacterial component 121 and a hydrocolloid contact component 122. The antibacterial component 121 comprises a substrate coated with alternating layers of PEI/Ag+ and PAA. The hydrocolloid contact component 122 comprises a hydrocolloid such as those known in the art as being particularly useful in wound dressings.

Process of Manufacturing Coating

The process for making the biologically active coating of the present invention comprises the steps of (1) dipping the substrate into an aqueous cationic polyelectrolyte solution, (2) rinsing the substrate with water, (3) drying the layer of cationic polymer (4) dipping the substrate into an aqueous anionic polyelectrolyte solution, (5) rinsing the substrate with water, (6) drying the deposited anionic polymer, (7) repeating the steps 1-6 to produce a multilayer biologically active film on the substrate. In one embodiment, a polar solvent other than water is used to deposit the organic material and to rinse the deposited layer.

Prior to dipping the substrate into the aqueous cationic polyelectrolyte solution, the substrate may be rinsed with methanol and then washed with water. Optionally, the substrate may be surface treated to improve the adhesion of the cationic polymer layer.

In one embodiment, the aqueous cationic polyelectrolyte solution comprises a solution of about 0.05% to about 1.5% by weight of cationic polymer. In one embodiment, the cationic polyelectrolyte solution comprises a solution of about 1.0% by weight of cationic polymer. The thickness of each organic polymer layer is generally less than about 200 nanometers. In one embodiment, the thickness is less than about 100 nanometers. In one embodiment, the thickness is of each organic layer is within the range of about 5 nanometers to about 60 nanometers. In another embodiment, the thickness of each organic layer is within the range of about 15 nanometers to about 50 nanometers.

The immersion time of the substrate in each of the coating solutions may be varied according to the particular coating solution, substrate composition, coating composition, or desired coating properties. The substrate may be held stationary in the coating solution, or the substrate may be moved within the coating solution bath, or may be continuously moved through the coating solution bath, for example, as a moving web of substrate material.

Test Methods:

The antimicrobial activity of the films of the present invention is evaluated using the Kirby-Bauer (Zone of Inhibition) and Dow Shake Flask (Log Reduction) test methods. The Kirby-Bauer test is conducted by placing the test article in contact with Agar containing 10⁵ colony forming units per ml. The Dow Shake Flask test is conducted by subjecting the test article to a flack containing test broth that is inoculated with 10⁵ colony forming units per ml. The number of viable microbes following 24 hours of contact with continuous agitation are quantified. This process is repeated every 24 hours using fresh organism until the targeted number of hours have been exhausted.

### EXAMPLES

### Example 1

An antimicrobial film is produced on a 7 mil corona-treated PET substrate by depositing multiple PEI-Ag⁺/PAA bilayers. The PET substrate is first immersed in PEI-Ag⁺ solution (1 mg/mL PEI; 20 millimolar (mM) AgNO₃) for 5 min. and then rinsed in water. The substrate is then immersed in a 3 mg/mL PAA solution for 5 min. and rinsed again in water. Multilayers are obtained by repetitive deposition of PEI-Ag⁺ and PAA. For deposition of bilayers subsequent to the first bilayers, the immersion time is about 1 minute. Antimicrobial films made up of 2-50 bilayers are produced.

The antimicrobial activity of the films is evaluated using the Kirby-Bauer test, which places the film in contact with Agar containing 10⁵ colony forming units per mL. The zone of inhibition of the films is about 1 mm to about 3 mm s. aureus after 24 hours.

### Example 2

An antimicrobial film is produced on a 7 mil PET substrate by depositing multiple PEI-Ag⁺/PAA bilayers alternating with multiple “inactive” barrier PDDA/clay bilayers. The PET substrate is first immersed in PEI-Ag⁺ solution (1 mg/mL PEI; 20 mM AgNO₃) for 5 min. and then rinsed in water. The substrate is then immersed in a 3 mg/mL PAA solution for 5 min. and rinsed again in water. Six active bilayers are obtained by repetitive deposition of PEI-Ag⁺ and PAA. The coated substrate is then immersed in a cationic solution of PDDA (3 mg/mL), rinsed and immersed in an anionic solution of sodium montmortillonite (3 mg/mL), and rinsed again in water. Six inactive bilayers are obtained by repetitive deposition of PDDA/clay. The antimicrobial film consists of 7 alternating blocks of 6 active and inactive bilayers (total of 42 bilayers).

### Example 3

An antimicrobial film is produced on a 7 mil PET substrate by depositing multiple PEI-cetrimide/PAA bilayers. The PET substrate is first immersed in PEI-cetrimide solution (1 mg/mL PEI; 20 mM cetrimide) for 5 min. and then rinsed in water. The substrate is then immersed in a 3 mg/mL PM solution for 5 min. and rinsed again in water. Multilayers are obtained by repetitive deposition of PEI-cetrimide and PAA. Antimicrobial films made up of 16 bilayers are produced.

The zone of inhibition of the antimicrobial film, evaluated using the Kirby-Bauer test, measures 8 mm to about 10 mm for s. aureus and 1 mm to about 4 mm for E. coli after 24 hours.

### Example 4

An antimicrobial film is produced on a 7 mil PET substrate by depositing multiple PEI-Ag⁺/PAA bilayers and multiple PEI-cetrimide/PAA bilayers. The PET substrate is first immersed in PEI-Ag⁺ solution (1 mg/mL PEI; 20 mM AgNO₃) for 5 min. and then rinsed in water. The substrate is then immersed in a 3 mg/mL PAA solution for 5 min. and rinsed again in water. Eight bilayers are obtained by repetitive deposition of PEI-Ag⁺ and PAA. The coated PET substrate is then immersed in PEI-cetrimide solution (1 mg/mL PEI; 20 mM cetrimide) for 5 min. and rinsed in water, followed by immersion in a 3 mg/mL PAA solution for 5 min. and rinsing in water. Eight bilayers are obtained by repetitive deposition of PEI-cetrimide and PAA. Antimicrobial films containing both Ag⁺ and cetrimide having a total of 16 bilayers are produced.

The zone of inhibition of the antimicrobial film, evaluated using the Kirby-Bauer test, measures 6 mm to about 9 mm for s. aureus and 1 mm to about 3 mm for E. coli after 24 hours.

### Example 5

An antimicrobial film is produced on a 7 mil PET substrate by depositing multiple PEI-cetrimide/PAA bilayers and multiple PEI/cephalosporin-PAA bilayers. The PET substrate is first immersed in PEI-cetrimide solution (1 mg/mL PEI; 20 mM cetrimide) for 5 min. and rinsed in water, followed by immersion in a 3 mg/mL PAA solution for 5 min. and rinsing in water. Eight bilayers are obtained by repetitive deposition of PEI-cetrimide and PAA. The coated substrate is then immersed in PEI solution (1 mg/mL), rinsed and then immersed in a cephalosporin-PAA solution (5 mM cephalosporin; 1 mg/mL PM) for 5 min. and rinsed again in water. Eight bilayers are obtained by repetitive deposition of PEI and cephalosporin-PAA. Antimicrobial films containing both cetrimide and cephalosporin having a total of 16 bilayers are produced.

### Example 6

An antimicrobial film is produced on a polyurethane foam substrate having a thickness of 0.625 inch (1.59 cm) by depositing multiple PEI-Ag⁺/PAA bilayers. The foam substrate is first immersed in PEI-Ag⁺ solution (1 mg/mL PEI; 20 mM AgNO₃) for 5 min. and then rinsed in water. The foam substrate is then immersed in a 3 mg/mL PAA solution for 5 min. and rinsed again in water. Multilayers are obtained by repetitive deposition of PEI-Ag⁺ and PAA. Antimicrobial films made up of 16 bilayers are produced. The 16 bilayer foam results in a 5 log reduction of microbial population within the first 2 hours of contact and is sustained for 72 hours.

### Example 7

A controlled release antimicrobial film is produced on a polyurethane foam substrate by depositing multiple PEI-cetrimide/PAA bilayers onto the substrate. The foam substrate is first immersed in a PEI-Cetrimide solution (1 mg/ml PEI; 20 mM cetrimide) for 5 minutes and then rinsed in water. The foam substrate is then immersed in a 3 mg/ml PAA solution for 5 minutes and rinsed again in water. Eight bilayers are obtained by repetitive deposiiton of PEI-Cetrimide and PAA. The coated substrate is then immersed in a PEI solution (1 mg/ml) for 5 minutes, rinsed and then immersed in a PAA solution (3 mg/ml) for 5 minutes and rinsed again in water. Four bilayers are obtained by repetitive deposition of PEI and PAA. The coated substrate is immersed in PEI-Ag+ solution (1 mg/ml of PEI; 20 mM AgNO₃) for 5 minutes and rinsed in water. The substrate is then immersed in a PAA solution (3 mg/ml PM) for 5 minutes and rinsed again in water. Eight bilayers are obtained by repetitive deposition of PEI-Ag+ and PAA. Multifunctional antimicrobial films made up of 20 bilayers are produced.

### Example 8

A controlled release antimicrobial film is produced on a polyurethane foam substrate by depositing multiple PEI-Ag+/PAA bilayers onto the substrate. The foam substrate is first immersed in a PEI-Ag+ solution (1 mg/ml PEI; 20 mM AgNO₃) for 5 minutes and then rinsed in water. The foam substrate is then immersed in a 3 mg/ml PAA solution for 5 minutes and rinsed again in water. Eight bilayers are obtained by repetitive deposiiton of PEI-Ag+ and PAA. The coated substrate is then immersed in a PEI solution (1 mg/ml) for 5 minutes, rinsed and then immersed in a PAA solution (3 mg/ml) for 5 minutes and rinsed again in water. Four bilayers are obtained by repetitive deposition of PEI and PAA. The coated substrate is immersed in PEI solution (1 mg/ml of PEI) for 5 minutes and rinsed in water. The substrate is then immersed in a cephalosporin-PAA solution (3 mg/ml PAA; 5 mM cephalosproin) for 5 minutes and rinsed again in water. Four bilayers are obtained by repetitive deposition of PEI and cephalosporin-PAA solution. The coated substrate is immersed in PEI-cetrimide solution (1 mg/ml of PEI; 20 mM cetrimide) for 5 minutes and rinsed in water. The substrate is then immersed in a PAA solution (3 mg/ml PM) for 5 minutes and rinsed again in water. Eight bilayers are obtained by repetitive deposition of PEI and PAA. Multifunctional antimicrobial films made up of 24 bilayers are produced.

### Example 9

A controlled release antimicrobial film having a biocompatibility layer is produced by depositing multiple PEI-chitosan/PSS bilayers onto a polyurethane foam substrate. The foam substrate is first immersed in a PEI-chitosan solution (1 mg/ml PEI; 20 mM chitiosan) for 5 minutes and then rinsed in water. The foam substrate is then immersed in a 3 mg/ml PSS solution for 5 minutes and rinsed again in water. Four bilayers are obtained by repetitive deposition of PEI-chitosan and PSS layers. The coated substrate is then immersed in a PEI solution (1 mg/ml) for 5 minutes, rinsed and then immersed in a PAA solution (3 mg/ml) for 5 minutes and again in water. Four bilayers are obtained by repetitive deposition of PEI and PAA. The coated substrate is then immersed in a PAA solution (3 mg/ml PAA) for 5 minutes and rinsed again in water. Eight bilayers are obtained by repetitive deposition of PEI-Ag+ and PAA. A 16 bilayer film having antimicrobial and biocompatible blocks are produced.

### Example 10

A biocompatibility film is produced on a polyurethane foam substrate by depositing multiple PEI-chitosan/PSS bilayers onto the foam. The foam substrate is first immersed in a PEI-chitosan solution (1 mg/ml; 20 mM chitosan) for 5 minutes and then rinsed in water. The foam substrate is then immersed in a 3 mh/ml PSS solution for 5 minutes and rinsed again in water. Four bilayers are obtained by repetitive deposition of PEI-chitosan and PSS.

Although the invention has been shown and described with respect to a certain embodiment or embodiments, it is obvious that equivalent alterations and modifications will occur to others skilled in the art upon reading and understanding of this specification. In particular regard to the various functions performed by the above described elements (components, assemblies, compositions, etc.), the terms used to describe such elements are intended to correspond, unless otherwise indicated, to any element that performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A multilayer antimicrobial coating on a substrate comprising alternating layers of at least one cationic material and at least one anionic material; and having at least one biologically active agent complexed with the cationic or anionic material; wherein the thickness of each of the cationic and anionic layers is less than about 200 nanometers.

2. The multilayer antimicrobial coating of claim 1 wherein the substrate comprises a flexible substrate.

3. The multilayer coating of claim 1 wherein the substrate comprises a polymeric material.

4. The multilayer coating of claim 3 wherein the substrate is a corona treated polymeric film.

5. The multilayer coating of claim 3 wherein the substrate comprises a polymeric foam.

6. The multilayer coating of claim 1 wherein the substrate comprises a thin film dressing.

7. The multilayer coating of claim 1 wherein the biologically active agent is complexed with the cationic layer.

8. The multilayer coating of claim 1 wherein the biologically active agent is complexed with the anionic layer.

9. The multilayer coating of claim 7 wherein the biologically active agent comprises silver ions.

10. The multilayer coating of claim 7 wherein the biologically active agent comprises cetrimide.

11. The multilayer coating of claim 7 wherein the biologically active agent comprises silver ions in combination with cetrimide.

12. The multilayer coating of claim 8 wherein the biologically active agent comprises cephalosporin.

13. The multilayer coating of claim 1 wherein the cationic material comprises polyethyleneimine.

14. The multilayer coating of claim 1 wherein the anionic material comprises poly(sodium 4-styrenesulfonate).

15. The multilayer coating of claim 1 wherein the anionic material comprises poly(acrylic acid).

16. The multilayer coating of claim 1 wherein the substrate and the coating are substantially transparent.

17. The multilayer coating of claim 1 wherein the cationic material or the anionic material or both comprises a crosslinkable polyelectrolyte.

18. The multilayer coating of claim 1 wherein the coating comprises 2 to 100 bilayers of cationic and anionic layers.

19. The multilayer coating of claim 1 wherein the coating comprises 4 to 50 bilayers of cationic and anionic layers.

20. The multilayer coating of claim 1 wherein the coating comprises 4 to 35 bilayers of cationic and anionic layers.

21. The multilayer coating of claim 1 wherein the substrate comprises an adhesive coated polymeric film.

22. The multilayer coating of claim 1 further comprising a barrier coating within the multilayer coating, wherein the barrier coating comprises alternating layers of biologically inactive cationic and anionic layers.

23. The multilayer coating of claim 22 wherein the biologically inactive anionic layer comprises an inorganic material.

24. The multilayer coating of claim 23 wherein the inorganic material comprises silicate clay.

25. The multilayer coating of claim 1 further comprising a sustained-release coating within the multilayer coating, wherein the sustained release coating comprises alternating layers of biologically inactive cationic and anionic layers.

26. The multilayer coating of claim 25 wherein the biologically inactive anionic layer comprises poly(acrylic acid), poly(sodium 4-styrenesulfonate) or combinations thereof.

27. The multilayer coating of claim 25 wherein the biologically inactive cationic layer comprises polyethyleneimine.

28. The multilayer coating of claim 1 further comprising a layer of hydrogel overlying the alternating layers of cationic and anionic material.

29. The multilayer coating of claim 1 further comprising a pattern-coated adhesive layer overlying the alternating layers of cationic and anionic material.

30. A wound dressing comprising a multilayer coating wherein the multilayer coating comprises alternating layers of at least one cationic material and at least one anionic material; and has at least one biologically active agent complexed with the cationic or anionic material; wherein the thickness of each of the cationic and anionic layers is less than about 200 nanometers.

31. The wound dressing of claim 30 further comprising a hydrogel.

32. The wound dressing of claim 30 further comprising a hydrocolloid.

33. The wound dressing of claim 30 further comprising a sustained release coating within the multilayer coating.
